(19) United States
(12) Patent Application Publication
(10) Pub. No.: US 2019/0021593 A1
(43) Pub. Date: Jan. 24, 2019

(54) ELECTRONIC APPARATUS AND METHOD OF CONTROLLING THE SAME

(71) Applicant: Samsung Electronics Co., Ltd., Suwon-si, Gyeonggi-do (KR)

(72) Inventors: Hyun-suk KWAK, Seoul (KR); Jun-ho KOH, Suwon-si (KR); Chang-hyun KIM, Seoul (KR); Yong-chan LEE, Seoul (KR); Jeong-eun LEE, Seongnam-si (KR)

(73) Assignee: Samsung Electronics Co., Ltd., Suwon-si, Gyeonggi-do (KR)

(21) Appl. No.: 16/068,788

(22) PCT Filed: Jan. 5, 2017
(86) PCT No.: PCT/KR2017/000165

§ 371 Date: Jul. 9, 2018

(30) Foreign Application Priority Data
Jan. 18, 2016 (KR) 10-2016-0006065

(51) Int. Cl. A61B 5/00; A61B 5/08; A61B 7/00

(52) U.S. Cl. CPC A61B 5/0004; A61B 5/742; A61B 7/003; A61B 5/0816

(57) ABSTRACT

The present invention relates to an electronic apparatus comprising a communicator configured to communicate with a plurality of information collection modules for collecting physical condition information at a plurality of points on a body of a user; and a controller configured to select a second point among the plurality of points based on first physical condition information received from a first information collection module corresponding to a first point among the plurality of points on the body, and control the communicator to receive second physical condition information from second information collection module corresponding to the selected second point. Thus, it is possible to prevent time from being wasted due to considering information about physical conditions irrelevant to diagnostic items.

### TECHNICAL FIELD

The present invention relates to an electronic apparatus and a method of controlling the same.

### BACKGROUND ART

With recent development of a portable electronic apparatus, various technologies have been developed to diagnose a user's physical conditions through the portable electronic apparatus. In particular, the portable electronic apparatus may interwork with an external sensing apparatus attached to a plurality of points on a body of a user to collect information about a lung sound of the user and analyze the collected information, thereby diagnosing a respiratory disease.

However, when the portable electronic apparatus diagnoses the respiratory disease by analyzing the whole information about the lung sounds collected from the plurality of points on the body of the user, it takes a long time to diagnose the respiratory disease since information about a lung sound related to a respiratory disease that has not occurred in the user has to be also collected and analyzed.

### DISCLOSURE

### Technical Problem

Accordingly, an object of the present invention is to provide an electronic apparatus and a control method thereof, which can prevent time from being wasted due to considering information about physical conditions irrelevant to diagnostic items.

### Technical Solution

The object of the present invention is achieved by providing an electronic apparatus comprising: a communicator configured to communicate with a plurality of information collection modules for collecting physical condition information at a plurality of points on a body of a user; and a controller configured to select a second point among the plurality of points based on first physical condition information received from a first information collection module corresponding to a first point among the plurality of points on the body, and control the communicator to receive second physical condition information from second information collection module corresponding to the selected second point.

Here, the controller may determine at least one diagnostic item among a plurality of diagnostic items based on the received first physical condition information.

Here, the controller selects the second point at which the physical condition information is collectable corresponding to the at least one determined diagnostic item.

Further, the electronic apparatus may further comprise an output part configured to output diagnosis information, wherein the controller controls the output part to output the diagnosis information about the at least one determined diagnostic item based on the received second physical condition information.

Here, the controller may determine the at least one diagnostic item in accordance with whether the received first physical condition information involves information about characteristics corresponding to the diagnostic items.

Here, the physical condition information may include the information about the lung sound.

Here, the diagnostic item may include at least one among respiratory diseases.

Here, the characteristic corresponding to each diagnostic item may comprise at least one among unique loudness, loudness variation, a frequency, a cycle, sustainability, sound quality, a tone, tone variation, intensity, intensity variation, relative time in inspiratory and expiratory stages, and strength of each diagnostic item.

Here, the diagnosis information may include a pathogenic degree of the respiratory disease.

Here, the output part may comprise a display, and the controller may control the display to display the diagnosis information.

The object of the present invention is achieved by providing a method of controlling an electronic apparatus communicating with a plurality of information collection modules that collect physical condition information at a plurality of points on a body of a user, the method comprising: receiving first physical condition information from a first information collection module corresponding to a first point among the plurality of points on the body; selecting a second point among the plurality of points based on the received first physical condition information; and receiving second physical condition information from a second information collection module corresponding to the selected second point.

Here, the method may further comprise determining at least one diagnostic item among a plurality of diagnostic items based on the received first physical condition information.

Here, the selecting may comprise selecting the second point at which the physical condition information is collectable corresponding to the at least one determined diagnostic item.

Here, the method may further comprise outputting the diagnosis information about the at least one determined diagnostic item based on the received second physical condition information.

Here, the determining may comprise determining the at least one diagnostic item in accordance with whether the received first physical condition information involves information about characteristics corresponding to the diagnostic items.

Here, the physical condition information may include the information about the lung sound.

Here, the diagnostic item may include at least one among respiratory diseases.

Here, the characteristic corresponding to each diagnostic item may comprise at least one among unique loudness, loudness variation, a frequency, a cycle, sustainability, sound quality, a tone, tone variation, intensity, intensity variation, relative time in inspiratory and expiratory stages, and strength of each diagnostic item.

Here, the diagnosis information may include a pathogenic degree of the respiratory disease.

Here, the outputting may comprise displaying the diagnosis information on a display.

The object of the present invention is achieved by providing a recording medium for storing a computer program comprising a code which implements a method of controlling an electronic apparatus as a computer readable code, wherein operations of an electronic apparatus that communicates with a plurality of information collection modules for collecting physical condition information at a plurality of points on a body of a user, the operations comprising: receiving first physical condition information from a first information collection module corresponding to a first point among the plurality of points on the body; selecting a second point among the plurality of points based on the received first physical condition information; and receiving second physical condition information from a second information collection module corresponding to the selected second point.

### Advantageous Effects

According to the present invention, there are provided an electronic apparatus and a control method thereof, which can prevent time from being wasted considering information about physical conditions irrelevant to diagnostic items.

### DESCRIPTION OF DRAWINGS

FIG. 1 illustrates a diagnosis system for physical conditions according to one embodiment of the present invention.

FIG. 2 illustrates an information collector according to one embodiment of the present invention.

FIG. 3 illustrates a process of diagnosing physical conditions according to one embodiment of the present invention.

FIG. 4 illustrates a process of diagnosing physical conditions according to one embodiment of the present invention.

FIG. 5 is a flowchart of diagnosing physical conditions according to one embodiment of the present invention.

FIG. 6 illustrates an electronic apparatus according to one embodiment of the present invention.

FIG. 7 is a block diagram of a diagnosis system for physical conditions according to one embodiment of the present invention.

FIG. 8 illustrates a process of diagnosing physical conditions according to another embodiment of the present invention.

FIG. 9 illustrates a process of diagnosing physical conditions according to another embodiment of the present invention.

FIG. 10 illustrates a process of diagnosing physical conditions according to one embodiment of the present invention.

FIG. 11 illustrates a process of diagnosing physical conditions according to another embodiment of the present invention.

### BEST MODE

Below, embodiments of the present invention will be described with reference to the accompanying drawings. In the embodiments set forth herein, only elements directly related to the idea of the present invention will be described, and descriptions about the other elements will be omitted. However, it will be appreciated that the elements, the descriptions of which are omitted, are necessary to materialize the apparatus or system to which the idea of the present invention is applied.

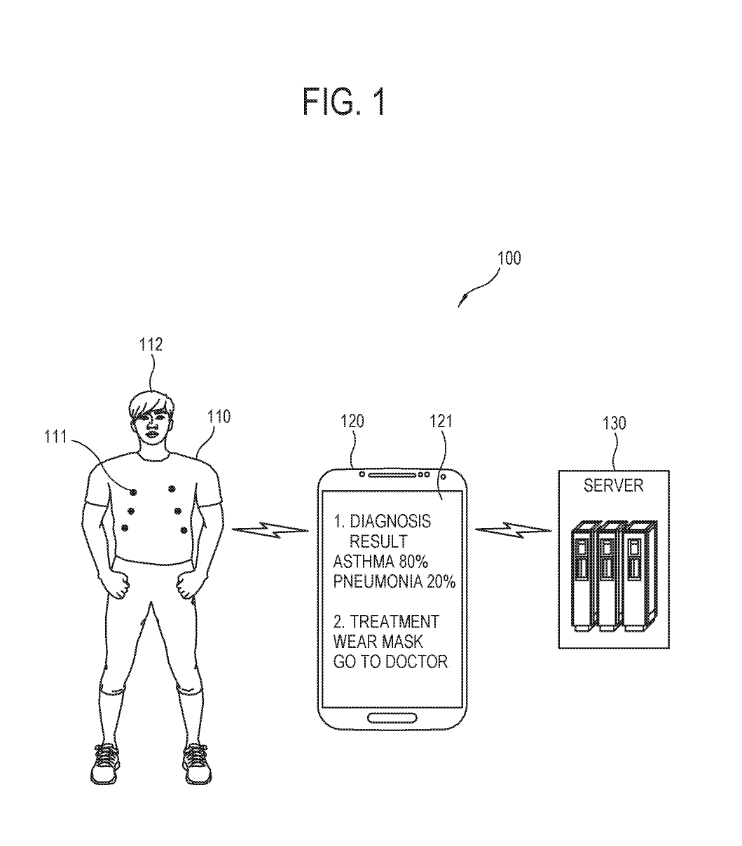

FIG. 1 illustrates a diagnosis system for physical conditions according to one embodiment of the present invention.

Referring to FIG. 1, a physical condition diagnosis system 100 according to one embodiment of the present invention may include an information collector 110, an electronic apparatus 120, and a server 130.

The information collector 110 may include a plurality of information collection modules 111 for collecting information about physical conditions of a user 112. The information collector 110 may be manufactured as clothes to be worn by a user 112, and the information collection module 111 may be provided in the form of a patch or film that can be attached to and detached from the inside or outside of the information collector 110. Therefore, the information collection module 111 is capable of collecting information about a user's physical conditions as it is close to a body of a user 112.

In particular, the information about physical conditions collectable in the information collection module 111 may include information about a lung sound. That is, the information collection module 111 may include a sensor for collecting the lung sound to diagnose a respiratory disease of a user 112. However, the information collection module 111 is not limited to the collection of the lung sound for diagnosing the respiratory disease, and may be used for diagnosing at least one among a lung disease, presence of breathing, normality of breathing, a characteristic of breathing, a frequency of breathing, strength of breathing, stability of breathing, cardiac insufficiency.

The information collection module 111 may be provided at points corresponding to regions of a body from which a lung sound of a user is collectable. However, the information collection module 111 may be provided at different points corresponding to users in accordance with sex, age, height, and the like body conditions of a user 112.

The information collection module 111 may collect information about a lung sound from each individual point. Here, the lung sound may include not only a breading sound such as a vesicular breathing sound, a bronchial breathing sound, a bronchial vesicular breath sound, etc. but also a wheeze sound, a crackle sound, a pleural friction rub sound, a stridor, a guttural sound, a rhonchus sound, a voice of a user 112, and the like. Such a lung sound may have unique characteristics such as unique loudness, loudness variation, tone, tone variation, sound quality, frequency, cycle, sustainability, intensity, intensity variation, inspiratory and expiratory time, strength, etc. caused by a unique point of a body according to respiratory diseases.

The information collection module 111 may transmit such collected information about the lung sound to the electronic apparatus 120.

The electronic apparatus 120 may receive the information about the lung sound from the information collection module 111, and monitor the received information about the lung sound. Here, the information about the lung sound to be monitored may include not only information about the points where the lung sound is collected, but also information about whether the lung sound corresponds to a crackle sound, a wheeze sound, a pleural friction rub sound, a stridor, a guttural sound, a rhonchus sound, and a voice of a user 112. Further, the information about the lung sound may include information about the characteristic of the lung sound, such as the loudness, tone, sound quality, frequency, cycle, sustainability, loudness variation, tone variation, frequency variation, cycle variation, intensity, intensity variation and the like of the lung sound.

The electronic apparatus 120 may previously store reference information that includes information about a normal breathing sound and an abnormal breathing sound caused by various respiratory diseases. Therefore, the electronic apparatus 120 may compare the stored reference information with the information about the lung sound received from the information collection module 111.

For example, the reference information about pneumonia may be stored as a user's pronunciation of alphabet ‘E’ at an upper point of the lung sounds like that of alphabet ‘A’. Thus, the electronic apparatus 120 may compare the information about the lung sound received from the information collection module 111 provided at the upper point of the lung with the reference information about the pneumonia. Besides, the electronic apparatus 120 may store the reference information based on information about unique lung sounds caused by various respiratory diseases such as asthma, bronchitis, respiratory infections, pulmonary edema, congestive cardiac insufficiency, and bronchiectasis. However, the electronic apparatus 120 is not limited to only a diagnosis of a respiratory disease, and may store reference information about at least one among lung diseases, presence of breathing, normality of breathing, a characteristic of breathing, a frequency of breathing, strength of breathing, stability of breathing, and cardiac insufficiency.

Accordingly, the electronic apparatus 120 can diagnose a user's respiratory disease based on information about a lung sound received from the information collection module 111 of the information collector 110, and outputs a result of the diagnosis. In particular, the electronic apparatus 120 may output a pathogenic degree of a respiratory disease as a diagnosis result, which shows incidence, progress, infectivity, etc. about at least one respiratory disease based on intensity, strength, and the like information about the lung sound. Further, the electronic apparatus 120 may output a pathogenic degree of a respiratory disease of a user 112 as a diagnosis result, based on statistical probability of a respiratory disease due to sex, age, genetic factors, medical history, etc. of the user 112.

The electronic apparatus 120 may display a diagnosis result on a display 121. The displayed diagnosis result may not only show the stage of the respiratory disease but also additionally show preventive measures, emergency measures, relief measures and the like of the respiratory disease. Further, the electronic apparatus 120 may output diagnosis results and additional information in the form of a sound.

Below, a process of diagnosing physical conditions of a user 112 in the electronic apparatus 120 interworking with the information collector 110 according to one embodiment of the present invention will be described in detail.

According to this embodiment, the electronic apparatus 120 receives first physical condition information collected by a first information collection module provided at a first point among a plurality of body points of the user 112, and select a second point for collecting physical condition information among the plurality of body points based on the first physical condition information.

Here, the first physical condition information includes information for determining a physical condition of a user 112. The physical condition is caused by various diseases that may occur in the user 112, which may show not only a respiratory disease but also at least one among a lung disease, presence of breathing, normality of breathing, a characteristic of breathing, a frequency of breathing, strength of breathing, stability of breathing, and cardiac insufficiency. Further, the information for determining the physical condition refers to information about characteristics of unique lung sounds caused by various diseases, which may include loudness, tone, sound quality, frequency, cycle, sustainability, loudness variation, tone variation, frequency variation, cycle variation, intensity, intensity variation, etc. of the lung sound.

Meanwhile, the electronic apparatus 120 may be configured to store information about the points respectively corresponding to various diseases among the plurality of body points of the user 112. Here, the points respectively corresponding to various diseases may refer to unique points where the physical condition information is collected for diagnosing various diseases. At least one unique point may correspond to each disease, and the unique points may be shared between the diseases. Accordingly, the electronic apparatus 120 may select a plurality of second points based on the initial first physical condition information. In particular, when the plurality of second points are selected, the plurality of second points may be prioritized based on intensity, strength, etc. of the unique characteristics of various diseases included in the first physical condition information.

For example, it will be assumed that the electronic apparatus 120 determines a respiratory disease based on a lung sound. That is, the electronic apparatus 120 may monitor whether a plurality of pieces of information about a lung sound received from the plurality of information collection modules 111 corresponds to the reference information about a normal lung sound or the reference information about an abnormal lung sound, and monitors what disease the unique lung sound corresponds to in the case of corresponding to the reference information about the abnormal lung sound. When it is determined during the monitoring that the information about a first lung sound corresponds to the reference information about a certain respiratory disease, the electronic apparatus 120 may determine the certain respiratory disease as a respiratory disease that may occur in the user 112. Here, the respiratory disease that may occur is placed into a candidate group including a plurality of respiratory diseases. This is because the plurality of respiratory diseases are likely to have similar unique lung sounds. That is, the loudness of the lung sound collected from one point among the plurality of points on the body of the user 112 may include unique loudness that is caused by pneumonia and respiratory obstruction. In this case, the electronic apparatus 120 may determine pneumonia and the respiratory obstruction, which belong to the candidate group of the respiratory disease that may occur in the user, as the diagnostic items.

The electronic apparatus 120 may output the diagnosis information about the diagnostic item corresponding to the respiratory diseases that may occur. Here, when one diagnostic item is related to the respiratory disease that may occur, the electronic apparatus 120 may determine an attack probability of the diagnostic item. When a plurality of diagnostic items are related to the respiratory disease that may occur, the electronic apparatus 120 may determine an attack probability of each diagnostic item.

To this end, the electronic apparatus 120 may receive second physical condition information from second information collection modules provided at the second points corresponding to the diagnostic item among the plurality of points on the body. The electronic apparatus 120 is capable of storing information about the points of the body to be diagnosed with respect to each respiratory disease that may occur, and thus selecting the points of the body from which the second physical condition information is collectable for diagnosing the respiratory disease that may occur.

The electronic apparatus 120 may make a request for the second physical condition information to the information collection module 111 corresponding to the selected points of the body, and output the diagnosis information about the respiratory disease that occurs among the diagnostic items determined based on the second physical condition information.

In the foregoing cases, when pneumonia and the respiratory obstruction are determined for the diagnostic items as the respiratory disease that may occur, the electronic apparatus 120 may make a request for information about loudness of a lung sound to each information collection module 111 corresponding to each point, based on the information about the points of the body for diagnosing the pneumonia and the information about the points of the body for diagnosing the respiratory obstruction.

When the information about the loudness of the lung sound collected from the points for diagnosing the pneumonia involves information about the unique loudness of the pneumonia, the electronic apparatus 120 may output diagnosis information that the occurred respiratory disease is the pneumonia. When the information about the loudness of the lung sound collected from the points for diagnosing the respiratory obstruction involves information about the unique loudness of the pneumonia, the electronic apparatus 120 may output diagnosis information that the occurred respiratory disease is the respiratory obstruction.

However, when the information about the loudness of the lung sound involves both the information about the loudness of the pneumonia and the information about the loudness of the respiratory obstruction, the electronic apparatus 120 may make a request for the information about the loudness of the lung sound to the information collection module 111 corresponding to other points for diagnosing the pneumonia or the respiratory obstruction, and finally determine whether the respiratory diseases occurred in the user 112 is either of the pneumonia or the respiratory obstruction or both the pneumonia and the respiratory obstruction, based on the received information about the loudness of the lung sound. Since the electronic apparatus 120 does not take only the loudness of the lung sound into account when determining the pneumonia or the respiratory obstruction, but may consider at least one among the tone, the sound quality, the frequency, the cycle, the sustainability, the loudness variation, the tone variation, the frequency variation, the cycle variation, the intensity, and the intensity variation of the lung sound.

Further, the electronic apparatus 120 may output the attack probabilities about the pneumonia and the respiratory obstruction as incidences (%). In this case, the electronic apparatus 120 may take sex, age, idiosyncrasy, chronic diseases, medical history, etc. of the user 112 into account.

Like this, the electronic apparatus 120 in the physical condition diagnosis system 100 according to one embodiment interworks with the plurality of information collection modules 111, determines the diagnostic item based on the first physical condition information corresponding to the first points in the body, and outputs the diagnosis information about the diagnostic item determined by receiving the second physical condition information from the second information collection module at the second points of the body corresponding to the determined diagnostic item, thereby quickly and accurately outputting a diagnosis result with regard to the respiratory disease that may occur.

That is, it is possible to output the diagnosis result based on only the physical condition information related to the determined diagnostic item, thereby preventing time from being wastefully taken in considering the physical condition information irrelevant to the diagnostic item.

Meanwhile, the physical condition diagnosis system 100 according to this embodiment may further include the server 130. The server 130 may make up for a limited memory capacity of the electronic apparatus 120, and be used in sharing the determined diagnostic item and the output diagnosis information with an external diagnosis system.

In the case of making up for the limited memory capacity of the electronic apparatus 120, it may mean that the electronic apparatus 120 cannot or does not store the reference information to be compared with the information about the lung sound received from the information collection module 111. In this case, the electronic apparatus 120 may transmit the information about the lung sound, received from the information collection module 111, to the server 130. The server 130 determines whether the information about the lung sound received from the electronic apparatus 120 corresponds to reference information about a normal lung sound or reference information about an abnormal lung sound, and transmits a result of the determination to the electronic apparatus 120. The electronic apparatus 120 determines the diagnostic item based on the result of the determination from the server 130, and makes a request for the physical condition information to the information collection module of the points corresponding to the determined diagnostic item. The electronic apparatus 120 may transmit the physical condition information at the points corresponding to the diagnostic item to the server 130, and the server 130 may output the diagnosis information about the diagnostic item based on the physical condition information at the points corresponding to the diagnostic item. The server 130 may transmit the output diagnosis information to the electronic apparatus 120, and share the output diagnosis information with an external diagnosis system.

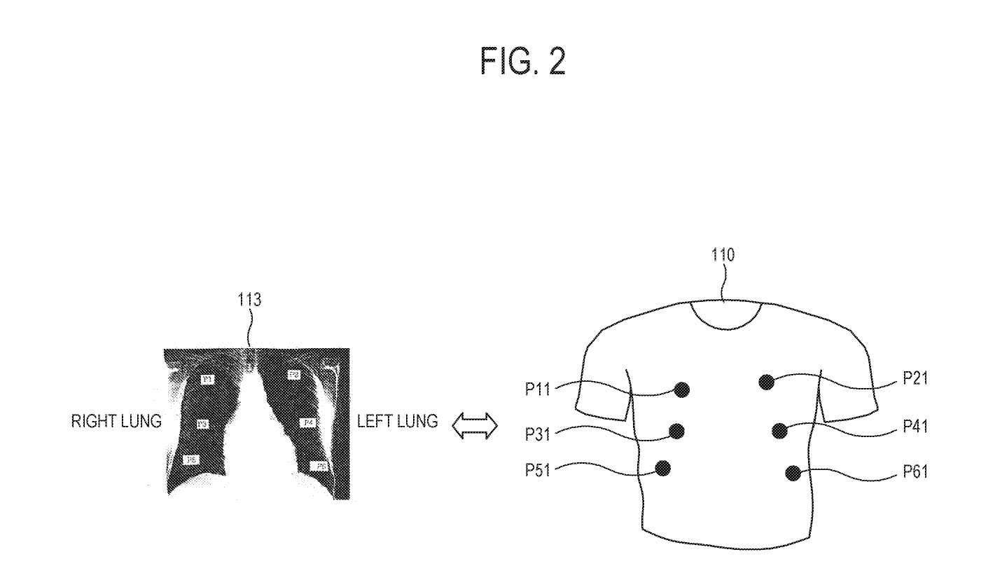

FIG. 2 illustrates an information collector according to one embodiment of the present invention. Referring to FIG. 2, the information collector 110 according to one embodiment of the present invention may include a plurality of information collection modules P11, P21, P31, P41, P51 and P61.

The plurality of information collection modules P11, P21, P31, P41, P51 and P61 may be provided in the points, at which at least one diagnostic item is determined based on the information about a user's physical conditions, among the plurality of points on the body of the user. For example, the plurality of information collection modules P11, P21, P31, P41, P51 and P61 may be provided respectively corresponding to the plurality of points P1, P2, P3, P4, P5 and P6 for determining a respiratory disease based on a lung sound generated from a user's lung 113 of a user. That is, the plurality of points P1, P2, P3, P4, P5 and P6 may refer to points at which the diagnostic item is determined based on the physical condition information collected from each point and the diagnosis information is output with regard to the diagnostic item.

Further, the plurality of information collection modules P11, P21, P31, P41, P51 and P61 may be provided at the points corresponding to a user's physical conditions. For example, in a case of a patient of chronic bronchitis, the information collection modules may be provided at a plurality of points at which the unique characteristics of the chronic bronchitis are collectable.

Below, a process of using the plurality of information collection modules to diagnose a user's physical conditions will be described in detail.

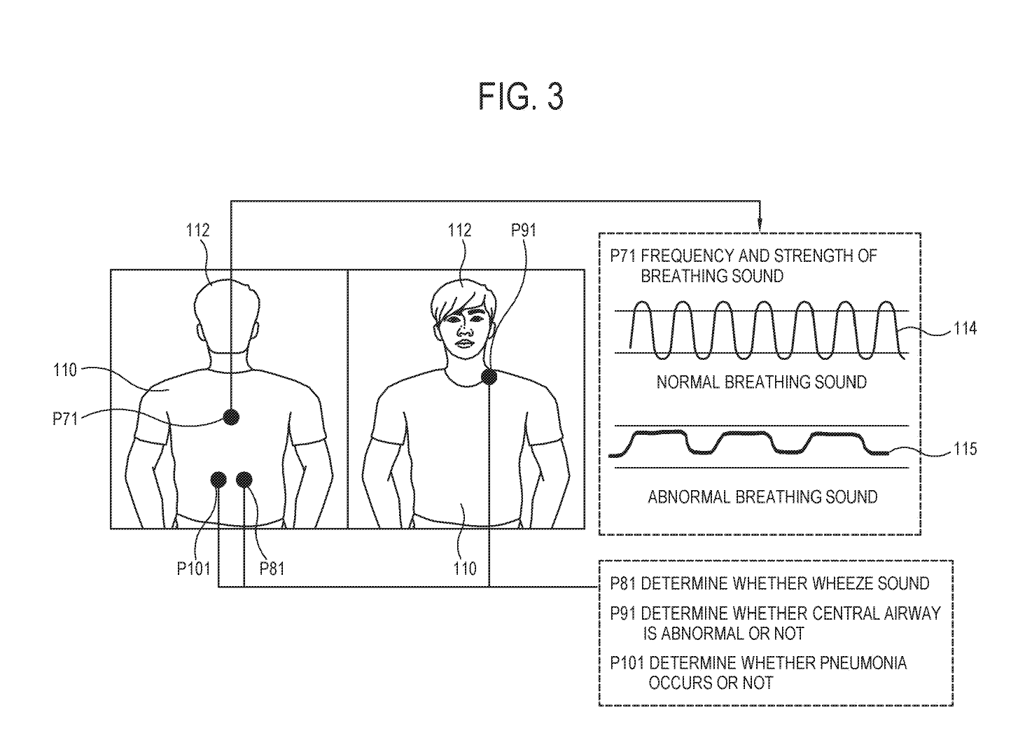

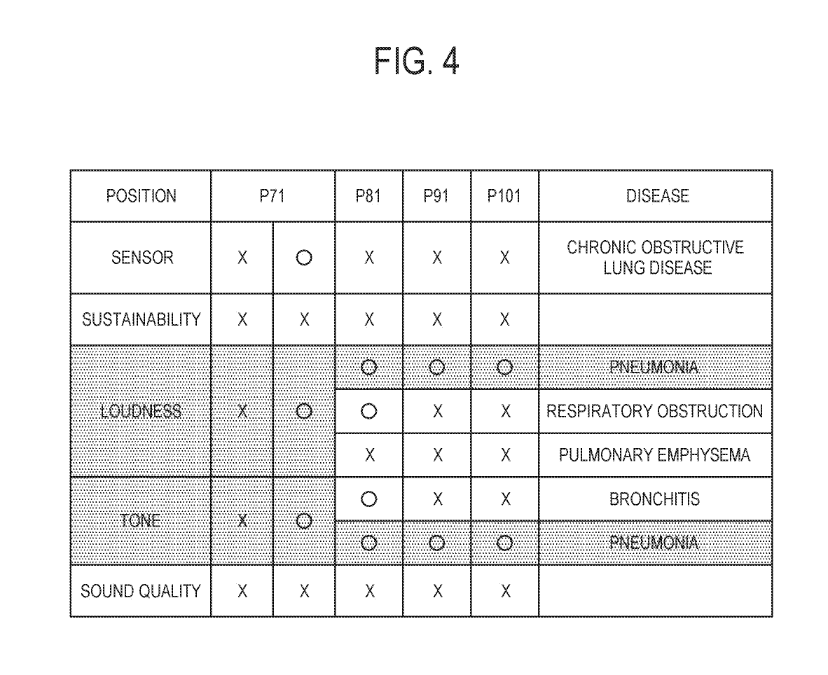

FIGS. 3 and 4 illustrate a process of diagnosing physical conditions according to one embodiment of the present invention. Referring to FIGS. 3 and 4, the electronic apparatus 120 according to this embodiment interworks with a plurality of information collection modules P71, P81, P91 and P101, thereby collecting and monitoring the information about the lung sound of the user 112 from the plurality of information collection modules P71, P81, P91 and P101.

The electronic apparatus 120 may determine not only chronic obstructive lung disease, pneumonia, the respiratory obstruction, pulmonary emphysema, and bronchitis as the respiratory disease that may occur, but also at least one among a lung disease, presence of breathing, normality of breathing, a characteristic of breathing, a frequency of breathing, strength of breathing, stability of breathing, and cardiac insufficiency as a diagnostic item, based on information about the frequency, loudness, tone, etc. of the lung sound collected in the information collection module P71 among the plurality of information collection modules P71, P81, P91 and P101.

That is, the electronic apparatus 120 is storing the reference information about the unique frequency, loudness, tone, tone variation, sound quality, frequency, cycle, sustainability, intensity, intensity variation, inspiratory and expiratory time, strength, etc. of the chronic obstructive lung disease, pneumonia, respiratory obstruction, pulmonary emphysema, and bronchitis, and therefore compares the information about the frequency, loudness, tone, etc. of the lung sound collected in the module P71 with the stored reference information, thereby determining whether the information about the frequency, loudness and tone of the lung sound collected in the module P71 includes the reference information about the unique frequency, loudness, tone, etc. of the chronic obstructive lung disease, pneumonia, respiratory obstruction, pulmonary emphysema, bronchitis, etc. Further, the electronic apparatus 120 is storing information about the information collection modules provided at the points for diagnosing the diseases among the plurality of information collection modules P71, P81, P91 and P101, and therefore selects the information collection module for diagnosing the disease determined based on the information about the frequency, loudness, tone, etc. of the lung sound collected in the module P71.

For example, in the case of the frequency, when the module P71 is storing the reference information about a frequency 114 of a normal breathing sound and collects information about a frequency 115 of an abnormal breathing sound, the electronic apparatus 120 may determine at least one respiratory disease as the diagnostic item in accordance with whether frequency information about an abnormal breathing sound involves the reference information about unique frequencies of various respiratory diseases. Besides, the electronic apparatus 120 is storing the information about the information collection modules provided at other points for diagnosing the determined diagnostic item except besides the module P71, and thus selects another information collection module provided in a different point corresponding to the determined diagnostic item.

Here, when a plurality of respiratory diseases is determined as the diagnostic items, the electronic apparatus 120 may determine order of diagnosing a respiratory disease that, which occur, based on the intensity, strength, etc. of the information about the frequency, loudness, tone, etc. of the lung sound collected in the module P71. Here, a diagnostic order of a respiratory disease may be given to preferentially collect the physical condition information from the information collection module corresponding to one among the plurality of respiratory diseases. Accordingly, the electronic apparatus 120 may preferentially receive the physical condition information from the information collection modules provided at the points corresponding to a respiratory disease having high priority among the plurality of respiratory diseases that may occur, and output diagnosis information about the respiratory disease based on the received physical condition information.

In the foregoing case, it will be assumed that the information about the frequency 115 of the abnormal breathing sound collected in the module P71 involves information about the unique frequencies of the pneumonia and the respiratory obstruction, and the information about the unique frequency of the pneumonia appears the most intensively. In this case, the electronic apparatus 120 may preferentially make a request for the information about the frequency of the lung sound to the module P81 capable of collecting the physical condition information about whether the pneumonia occurs or not, and output the diagnosis information about whether the pneumonia occurs or not, based on the information about the frequency of the lung sound received from the module P81.

Then, the electronic apparatus 120 can select the information collection module provided other points at which the physical condition information is collectable for diagnosing the respiratory obstruction.

In particular, the module P81 corresponds to the point for determining whether the pneumonia occurs or not based on a wheeze sound, and therefore the electronic apparatus 120 determines the occurrence of the pneumonia based on whether the wheeze sound from the module P81 involves information about the unique loudness, tone, etc. of the pneumonia.

However, the wheeze sound from the module P81 may involve not only the information about the unique loudness, tone, etc. of the pneumonia, but also the information about the unique loudness, tone, etc. of the respiratory obstruction. Even in this case, the electronic apparatus 120 may determine diagnostic order again in accordance with the intensity, strength, etc. of the information about the unique loudness, tone, etc. of the respiratory disease, involved in the wheeze sound from the module P81.

When it is sensed that the wheeze sound collected in the module P81 involves the intensity, strength, etc. of the information about the unique loudness, tone, etc. of the pneumonia the most intensively, the electronic apparatus 120 may output diagnosis information about the occurrence of the pneumonia based on the physical condition information collected at the modules P91 to P101 from which the physical condition information about the occurrence of the pneumonia is collectable.

In particular, in the case of using the physical condition information collected in the module P91, the electronic apparatus 120 may determine that a peripheral airway is abnormal when the wheeze sound from the module P91 has a frequency equal to or higher than 400 Hz, and may determine that a central airway is abnormal when the wheeze sound from the module P91 has a frequency lower than 400 Hz. Accordingly, the electronic apparatus 120 may determine that the pneumonia occurs with inflammation of pulmonary alveoli and the peripheral airway, when the wheeze sound from the module P91 has the frequency equal to or higher than 400 Hz.

Like this, the electronic apparatus 120 according to this embodiment may determine a diagnostic item prioritized in accordance with the loudness, loudness variation, tone, tone variation, sound quality, frequency, cycle, sustainability, intensity, intensity variation, inspiratory and expiratory time, and strength of the lung sound collected by one information collection module among the plurality of information collection modules, and performs a preferential diagnosis with regard to the diagnostic item based on the physical condition information received from the information collection modules at the points corresponding to the diagnostic item in accordance with priorities.

Thus, the electronic apparatus 120 may preferentially perform a diagnosis with regard to a respiratory disease having a high attack probability.

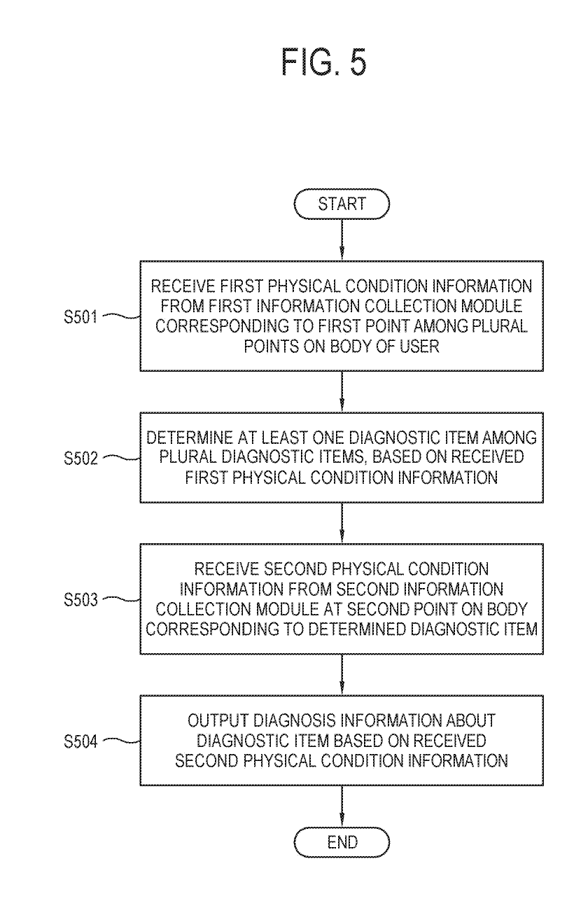

FIG. 5 is a flowchart of diagnosing physical conditions according to one embodiment of the present invention. A process of diagnosing the physical conditions according to this embodiment includes steps of receiving first physical condition information collected by a first information collection module provided at a first point among a plurality of body points of a user 112, and selecting a second point at which the physical condition information is collectable among the plurality of body points, based on the received first physical condition information.

In more detail, referring to FIG. 5 to describe the process of diagnosing the physical condition, the electronic apparatus 120 for diagnosing the physical condition according to this embodiment may receive the first physical condition information from the first information collection module corresponding to the first point among the plurality of points on the body of the user 112 (S501).

The electronic apparatus 120 may determine at least one diagnostic item among a plurality of diagnostic items, based on the received first physical condition information (S502). The electronic apparatus 120 may monitor whether the physical condition information received from the plurality of information collection modules corresponds to the reference information stored in the storage. Accordingly, the electronic apparatus 120 may determine at least one diagnostic item among a plurality of diagnostic items based on whether the received physical condition information corresponds to normal physical condition information or abnormal physical condition information.

For example, referring back to FIG. 2, the electronic apparatus 120 interwork with the information collector 110 of FIG. 2, and monitors the information about the lung sound received from the plurality of information collection modules P11, P21, P31, P41, P51 and P61, thereby determining that the information about the frequency of the first lung sound received from the module P31 corresponding to the point P3 involves information about a unique frequency of asthma. Accordingly, the electronic apparatus 120 may determine a diagnostic item that the respiratory disease that may occur is asthma, based on the result of the determination.

The electronic apparatus 120 may receive second physical condition information from a second information collection module at a second point on the body corresponding to the determined diagnostic item (S503). That is, when the diagnostic item is determined based on the first physical condition information, the electronic apparatus 120 may receive the second physical condition information from the second information collection module at the second point suitable for verifying the determined diagnostic item.

In the foregoing case, the electronic apparatus 120 may make a request for information about a frequency of a second lung sound to the module P51 corresponding to the point P5 capable of providing the information about the frequency of the second lung sound for verifying an attack probability of asthma, when a respiratory disease that may occur is determined for a diagnostic item as asthma on the basis of information about a frequency of a first lung sound received from the module P31 corresponding to the point P3.

The electronic apparatus 120 may output diagnosis information about the diagnostic item based on the received second physical condition information (S504). That is, the electronic apparatus 120 may determine the diagnosis information of the diagnostic item based on the second physical condition information suitable for verifying the attack probability of the determined diagnostic item. That is, the diagnosis information may be a result from verifying the diagnostic item.

In the foregoing case, the electronic apparatus 120 may receive the information about the frequency of the second lung sound from the module P51 capable of collecting the information about the frequency of the second lung sound suitable for verifying the attack probability of the asthma, when the respiratory disease that may occur is determined as the asthma on the basis of the information about the frequency of the first lung sound received from the module P31. The electronic apparatus 120 may output the diagnosis information, i.e. the asthma as an occurred respiratory disease, when the information about the frequency of the second lung sound involves the information about the unique frequency of the asthma.

When it is determined that the information about the frequency of the second lung sound does not involve the information about the unique frequency of the asthma, the electronic apparatus 120 may additionally receive information about a frequency of a lung sound collected from the information collection module corresponding to another point capable of verifying the attack probability of the asthma. The electronic apparatus 120 may output the diagnosis information about the asthma as the occurred respiratory disease, in accordance with whether the additionally received information about the frequency of the lung sound involves the information about the unique frequency of the asthma.

Meanwhile, the process of diagnosing the physical condition according to the present invention may be materialized by a recording medium in which a computer program is stored including a code for carrying out a control method of an electronic apparatus as a computer readable code.

Specifically, the control method of the electronic apparatus including the plurality of information collection modules for collecting information about physical conditions from a plurality of points on a body of a user may be achieved by a recording medium, in which a computer readable program is recorded, including: a step of receiving the first physical condition information from the first information collection module corresponding to the first point among the plurality of points on the body, a step of determining at least one diagnostic item among a plurality of diagnostic items based on the received first physical condition information, a step of receiving the second physical condition information from the second information collection module at the second point of the body corresponding to the determined diagnostic item, a step of outputting the diagnosis information about the diagnostic item based on the received second physical condition information.

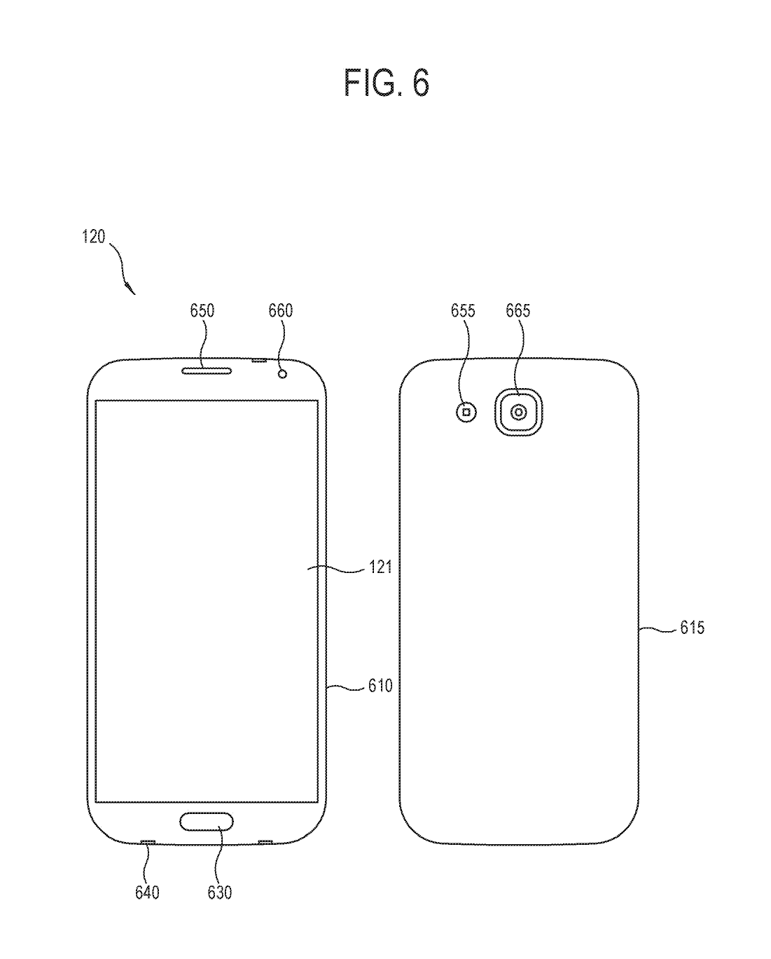

FIG. 6 illustrates an electronic apparatus according to one embodiment of the present invention. Referring to FIG. 6, the electronic apparatus 120 includes a front casing 610 and a rear casing 615, in which the front casing 610 and the rear casing 615 are configured to form an outer appearance of the electronic apparatus 120. Inside the front casing 610 and the rear casing 615, at least one middle casing may be additionally arranged.

The front casing 610 may be arranged with a display 121, a user input part 630, an audio receiver 640, a first audio output part 650, and a first image capturer 660. Further, the rear casing 615 may be arranged with a second audio output part 655 and a second image capturer 665.

The display 121 includes a liquid crystal display (LCD), an organic light emitting diode (OLED), or the like for visually displaying information. In the display 121, touch pads are overlapped having a layered structure. The display 121 operates as a touch screen to receive a user's input caused by the user's touch.

The user input part 630 may employ any tactile manner as long as it can allow a user to make control with tactile sensation. For example, the user input part 630 may be materialized by a dome switch or touch pad capable of receiving a command or information caused by a user's push or touch control, or may be materialized by a wheel or jog dial for rotating a key or by a joystick.

The user input part 630 may operate as a hot key for inputting a start, an end, a call, and the like command to the electronic apparatus 120, inputting selection or the like for an operation mode, or activating a specific function in the electronic apparatus 120. However, the function of the user input part 630 may be achieved through the display 121 operating as the touch screen.

The first image capturer 660 may be provided in the front of the electronic apparatus 120 and capture a front image. On the other hand, the second image capturer 665 may be provided in the back of the electronic apparatus 120 and capture a back image. Each of the first image capturer 660 and the second image capturer 665 may include a lens, an image sensor, etc.

The audio receiver 640 may be materialized by a microphone and adaptive to a user's voice, other sounds, etc.

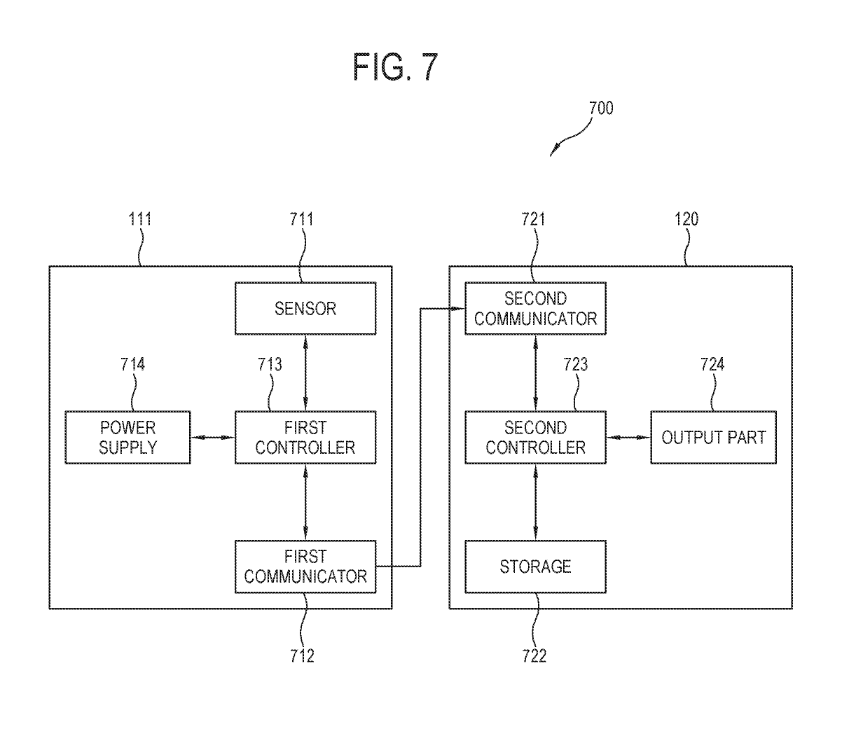

FIG. 7 is a block diagram of a diagnosis system for physical conditions according to one embodiment of the present invention. Referring to FIG. 7, a physical condition diagnosis system 700 according to one embodiment of the present invention may include the plurality of information collection modules 111 and the electronic apparatus 120.

The information collection module 111 may include a sensor 711, a first communicator 712, a first controller 713, and a power supply 714. However, these elements may be configured by integrating two or more elements into one element or dividing one element into two or more elements as necessary when they are put to practical use.

The sensor 711 may include a microphone capable of sensing a lung sound and the like physical condition information. For example, the sensor 711 can sense a lung sound having a specific frequency range of 100-500 Hz.

The first communicator 712 is provided to communicate with a second communicator 721 of the electronic apparatus 120. The first communicator 712 may transmit the physical condition information sensed by the sensor 711 to the electronic apparatus 120. In this case, the first communicator 712 may use Bluetooth, radio frequency identification (RFID), IrDA, ultra-wideband (UWB), ZigBee, etc. Further, the first communicator 712 may receive a control signal for operations of the information collection module 111 from the electronic apparatus 120.

The first controller 713 may be achieved by a central processing unit (CPU) or a processor, and control general operations of the elements in the information collection module 111. Further, the first controller 713 may control general operations of the elements in the information collection module 111 in response to the control signal of the electronic apparatus 120.

The power supply 714 may be materialized by a battery configured to supply power to the elements of the information collection module 111 and rechargeable with charging power from the outside. However, the battery can supply power to the elements of the information collection module 111 without receiving charging power from the outside since it may be rechargeable in accordance with a user's motion or body temperature.

The electronic apparatus 120 according to this embodiment may be a portable electronic apparatus such as a portable terminal, a mobile phone, a mobile pad, a media player, a tablet computer, a smart phone, or a personal digital assistant (PDA). The electronic apparatus 120 may include the second communicator 721, a storage 722, a second controller 723, and an output part 724.

The second communicator 721 is configured to communicate with the first communicator 712 of the information collection module 111, thereby receiving the physical condition information such as a lung sound from the first communicator 712, or transmitting a control signal for controlling the information collection module 111 to the first communicator 712.

The storage 722 may be configured to store the physical condition information received by the second communicator 721 and the reference information about physical conditions to be compared. The reference information may include the normal physical condition information and the abnormal physical condition information corresponding to various diseases.

When the physical condition information is received from the information collection module 111 corresponding to one point among the plurality of points on the body of the user 112, the second controller 723 compares the received physical condition information and the reference information stored in the storage 721. That is, the second controller 723 may perform comparison and determination with regard to whether the characteristics of the received physical condition information involve the characteristics of the reference information. In accordance with the comparison results, the second controller 723 may determine at least one diagnostic item corresponding to the physical condition information. The second controller 723 may receive the physical condition information from the information collection module 111 at the one point or at the points corresponding to the determined diagnostic item among the plurality of other points. In particular, the second controller 723 may prioritize the diagnostic items in accordance with intensity, strength, etc. of the physical condition information, and receive the physical condition information from the information collection module 111 corresponding to the diagnostic item in accordance with priorities.

The second controller 723 may output the diagnosis information about the diagnostic item based on the physical condition information received from the information collection module 111 at the points corresponding to the diagnostic item.

The output part 724 may output the diagnosis information of the second controller 723. The output part 724 may display information about diseases through the display 121, or output it as a sound through the audio output part 650, 655.

These elements may be configured by integrating two or more elements into one element or dividing one element into two or more elements as necessary when they are put to practical use. Further, the electronic apparatus 120 may further include a broadcast receiving module, a mobile communication module, a wireless Internet module, a near field communication module, a global positioning system (GPS) module, etc. as necessary.

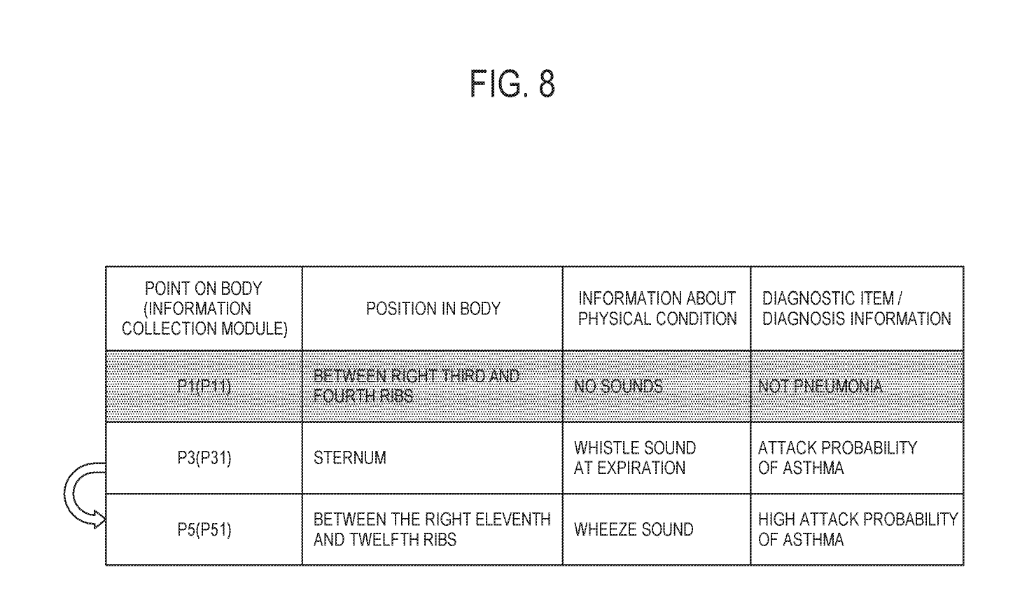

FIG. 8 illustrates a process of diagnosing physical conditions according to one embodiment of the present invention. The process of diagnosing the physical conditions according to one embodiment of the present invention may be achieved by interworking between the plurality of information collection modules 111 of FIG. 2, and the electronic apparatus 120 of FIG. 1.

In particular, the plurality of information collection modules P11, P21, P31, P41, P51 and P61 of FIG. 2 are provided corresponding to the plurality of points P1, P2, P3, P4, P5 and P6 on the body, and configured to collect the information about the lung sound from the corresponding point P1, P2, P3, P4, P5 and P6, respectively.

Referring to FIG. 7, the electronic apparatus 120 may receive the information about the lung sound collected by the module P11 among the plurality of information collection modules P11, P21, P31, P41, P51 and P61 through the second communicator 721. Here, the point P1 may be positioned between the right third and fourth ribs, and the information about the lung sound collected by the module P11 may involve information for determining whether pneumonia occurs or not. That is, the second controller 723 may determine whether pneumonia occurs or not based on the information about the lung sound collected by the module P11. To this end, the second controller 723 may compare the information about the lung sound collected by the module P11 with the reference information about the lung sound at the point P1. The reference information about the lung sound at the point P1 may refer to information previously stored in the storage 722. When the information about the lung sound at the point P1 involves information about no sounds, the output part 724 may output the diagnosis information of ‘not pneumonia’.

Meanwhile, the electronic apparatus 120 may receive the information about the lung sound from the module P31 corresponding to the point P3 of the body through the second communicator 721. Here, the point P3 may correspond to the sternum of the body, and the information about the lung sound collected by the module P31 may include information corresponding to a whistle sound generated at expiration. The electronic apparatus 120 may previously store the whistle sound, which is generated at the point P3, as the reference information about the pneumonia in the storage 722. Accordingly, when it is determined that the information about the lung sound collected by the module P31 involves the whistle sound generated at the expiration, the second controller 723 may determine that there is an attack probability of pneumonia.

The electronic apparatus 120 according to one embodiment of the present invention selects the point P5 for collecting the information about the lung sound suitable for verifying the attack, and receives the information about the lung sound collected by the module P51 corresponding to the point P5. Here, the point P5 may be positioned between the right eleventh and twelfth ribs, and the information about the lung sound received from the module P51 may involve wheeze sound information. Further, the reference information of the point P5 may refer to the information stored in the storage 722 as information showing the attack probability of the pneumonia when a wheeze sound is generated at the point P5. Accordingly, the second controller 723 may determine that the pneumonia occurs, when the information about the lung sound collected by the module P51 involves information corresponding to a wheeze sound.

The second controller 723 controls the output part 724 to output the diagnosis information about the pneumonia, thereby guiding a user 112 to properly take a measure against the diagnosed pneumonia.

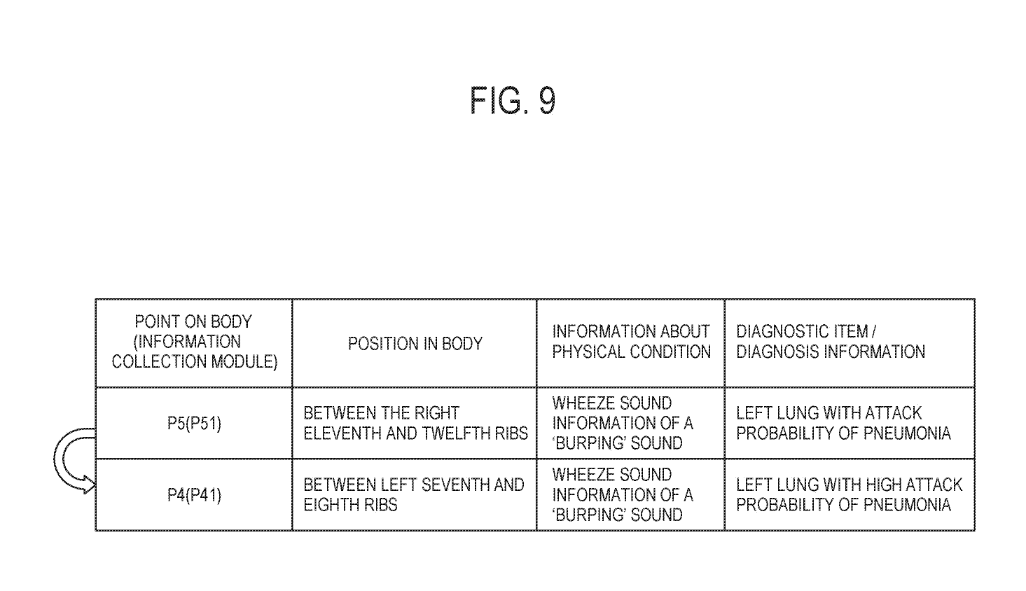

FIG. 9 illustrates a process of diagnosing physical conditions according to another embodiment of the present invention. Referring to FIG. 9, the electronic apparatus 120 according to this embodiment receives the information about the lung sound collected by the module P51 corresponding to the point P5 of the body from the module P51 through the second communicator 721. Here, the point P5 may be positioned between the right eleventh and twelfth ribs, and the information about the lung sound collected by the module P51 may involve information for determining whether pneumonia occurs or not. To this end, the second controller 723 compares the information about the lung sound collected by the module P51 with the reference information about the lung sound at the point P5. The reference information about the lung sound at the point P5 may refer to information previously stored in the storage 722, and include the reference information for determining that the pneumonia occurs in the left lung when the information about the lung sound at the point P5 involves wheeze sound information of a ‘burping’ sound

Accordingly, when it is determined that the information about the lung sound collected by the module P51 involves the wheeze sound information of the ‘burping’ sound, the second controller 723 may determine a diagnostic item for the attack probability of the pneumonia in the left lung.

Since it is determined based on the information about the lung sound collected by the module P51 that the left lung has the attack probability of the pneumonia, the second controller 723 selects the point P4 at which the information about the lung sound is collectable for verifying the attack probability of the pneumonia. That is, the electronic apparatus 120 is storing information about points on the body, at which the information about the lung sound has to be collected to determine the attack probability of the pneumonia, and reference information about a wheeze sound generated at the points by the pneumonia, in the storage 722. Accordingly, when a respiratory disease that may occur is determined as pneumonia based on the information about the lung sound from the module P51, the second controller 723 may select the point P4 capable of providing information about the pneumonia among the plurality of body points. Here, the point P4 may be positioned between the left seventh and eighth ribs, and the information about the lung sound collected by the module P41 corresponding to the point P4 may involve wheeze sound information of a ‘burping’ sound.

When it is determined that the information about the lung sound collected by the module P41 corresponding to the point P4 involves the wheeze sound information of the ‘burping’ sound, the second controller 723 may control the output part 724 to output diagnosis information that the left lung has a high attack probability of pneumonia.

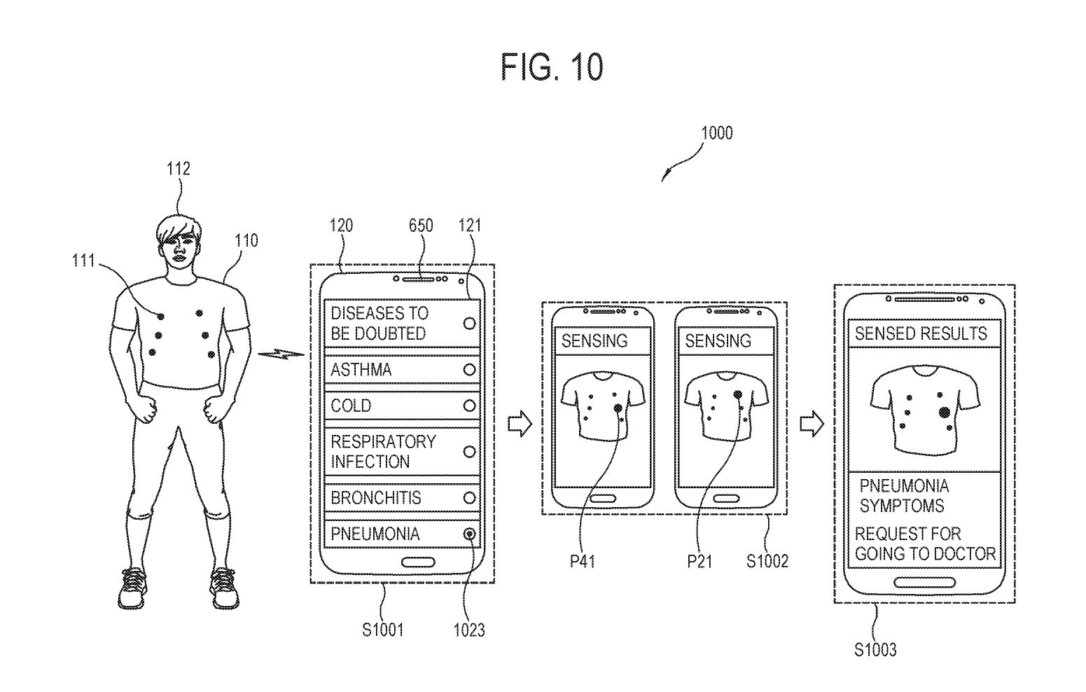

FIG. 10 illustrates a system for diagnosing physical conditions according to one embodiment of the present invention. Referring to FIG. 10, a diagnosis system 1000 according to one embodiment of the present invention includes the information collector 110 and the electronic apparatus 120. The information collector 110 may be manufactured to be worn on a user 112, and includes the plurality of information collection modules 111 provided at points on the body of the user 112 and collecting the physical condition information. The electronic apparatus 120 may include the display 121 and the audio output part 650.

The electronic apparatus 120 may display a plurality of applications for performing various functions through the display 121. The electronic apparatus 120 may execute an application, which is related to a diagnosis of physical conditions, among the plurality of applications in response to a user's selection. When the application for diagnosing the physical conditions is executed, the electronic apparatus 120 displays a plurality of diseases which can be diagnosed by the system for diagnosing the physical conditions (S1001). Accordingly, the user 112 may select a disease 1023 to be subjected to the diagnosis.

When the user 112 selects a disease, the electronic apparatus 120 collects physical condition information from at least one information collection module 111 capable of diagnosing whether the selected disease occurs or not among the plurality of information collection modules 111 (S1002). The electronic apparatus 120 is storing information about points, at which the occurrence of the selected disease is determinable, among the plurality of body points of the user 112, and information about the plurality of information collection modules 111 provided corresponding to the points. Further, the electronic apparatus 120 is storing information about diagnosis points for diagnosing each disease. Accordingly, the electronic apparatus 120 can sequentially receive the physical condition information from the information collection modules 111 at the diagnosis points corresponding to the selected disease. The electronic apparatus 120 may make the display 121 display the information collection module 111 which is collecting the physical condition information.

For example, the electronic apparatus 120 may previously store information about the module P41 corresponding to the point P4 and the module P21 corresponding to the point P2, which are capable of collecting the information about the lung sound for determining whether the pneumonia occurs or not. Accordingly, when the user 112 selects the diagnosis of the pneumonia 1023, pieces of information about the lung sounds collected in the modules P41 and P21 are sequentially received, and the attack probability of the pneumonia is determined based on the received information about the lung sounds.

When it is determined based on the received information about the lung sound that the pneumonia is likely to occur, the electronic apparatus 120 may output the determined diagnosis information through the display 121 or output it as a sound through the audio output part 650 (S1003).

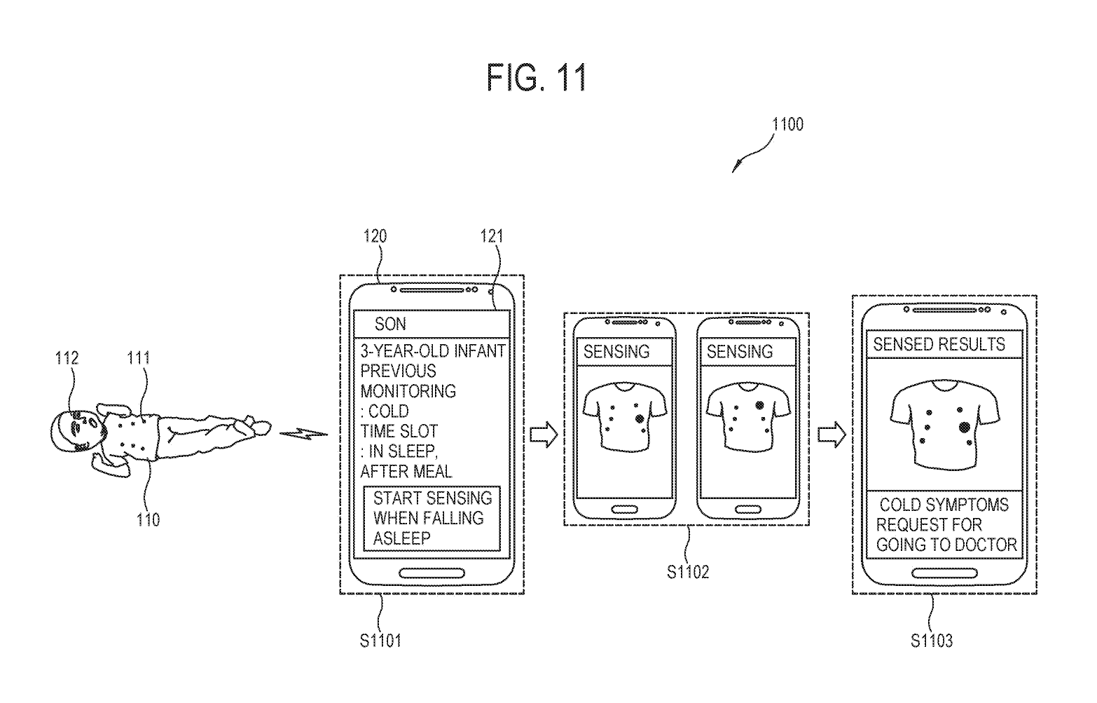

FIG. 11 illustrates a system for diagnosing physical conditions according to another embodiment of the present invention. Referring to FIG. 11, the diagnosis system 1000 according to one embodiment of the present invention may include the information collector 110 and the electronic apparatus 120.

A user 112 may set a disease desired to be diagnosed and a diagnosis time (S1101). In particular, it is very important for an infant, an old person, a chronic patient, etc. to previously diagnose a specific disease and take necessary measures against the disease since the disease is likely to quickly become serious. To this end, the electronic apparatus 120 according to this embodiment is configured to perform a diagnosis with regard to a preset disease at a preset diagnosis time. In particular, as shown in FIG. 11, the accuracy of the diagnosis information may be lowered since it is difficult to accurately collect the physical condition information in a timeslot during which an infant 112 has a lot of movement. Accordingly, a sleeping timeslot or a meal timeslot of an infant 112 may be designated as a diagnosis time, and a specific disease may be designated as a disease to be subjected to the diagnoses.

At the designated time, the electronic apparatus 120 interworks with the information collector 110 and collects the physical condition information through the information collection module 111 (S1102). Here, the electronic apparatus 120 is storing information about points for determining a progress of the designated disease among a plurality of body points of the infant 112, and information from the plurality of information collection modules 111 corresponding to the points. Further, the electronic apparatus 120 is storing information about the diagnosis points for diagnosing each disease. Accordingly, the electronic apparatus 120 may select the information collection module 111 corresponding to the diagnosis points for diagnosing the designated disease, and collect the physical condition information from the selected information collection module 111.

The electronic apparatus 120 determines whether the infant 112 catches the designated disease based on the collected physical condition information, and outputs the determination results (S1103).

Although the present invention has been described above in detail with preferred embodiments, the present invention is not limited to the foregoing embodiments but variously embodied within the appended claims.

1. An electronic apparatus comprising:
a communicator configured to communicate with a plurality of information collection modules for collecting physical condition information at a plurality of points on a body of a user; and
a controller configured to select a second point among the plurality of points based on first physical condition information received from a first information collection module corresponding to a first point among the plurality of points on the body, and control the communicator to receive second physical condition information from second information collection module corresponding to the selected second point.

2. The electronic apparatus according to claim 1, wherein the controller determines at least one diagnostic item among a plurality of diagnostic items based on the received first physical condition information.

3. The electronic apparatus according to claim 2, wherein the controller selects the second point at which the physical condition information is collectable corresponding to the at least one determined diagnostic item.

4. The electronic apparatus according to claim 3, further comprising an output part configured to output diagnosis information,
wherein the controller controls the output part to output the diagnosis information about the at least one determined diagnostic item based on the received second physical condition information.

5. The electronic apparatus according to claim 3, wherein the controller determines the at least one diagnostic item in accordance with whether the received first physical condition information involves information about characteristics corresponding to the diagnostic items.

6. The electronic apparatus according to claim 5, wherein the characteristic corresponding to each diagnostic item comprises at least one among unique loudness, loudness variation, a frequency, a cycle, sustainability, sound quality, a tone, tone variation, intensity, intensity variation, relative time in inspiratory and expiratory stages, and strength of each diagnostic item.

7. The electronic apparatus according to claim 4, wherein the output part comprises a display, and
the controller controls the display to display the diagnosis information.

8. A method of controlling an electronic apparatus communicating with a plurality of information collection modules that collect physical condition information at a plurality of points on a body of a user, the method comprising:
receiving first physical condition information from a first information collection module corresponding to a first point among the plurality of points on the body;
selecting a second point among the plurality of points based on the received first physical condition information; and
receiving second physical condition information from a second information collection module corresponding to the selected second point.

9. The method according to claim 8, further comprising determining at least one diagnostic item among a plurality of diagnostic items based on the received first physical condition information.

10. The method according to claim 9, wherein the selecting comprises selecting the second point at which the physical condition information is collectable corresponding to the at least one determined diagnostic item.

11. The method according to claim 10, further comprising outputting the diagnosis information about the at least one determined diagnostic item based on the received second physical condition information.

12. The method according to claim 10, wherein the determining comprises determining the at least one diagnostic item in accordance with whether the received first physical condition information involves information about characteristics corresponding to the diagnostic items.

13. The method according to claim 12, wherein the characteristic corresponding to each diagnostic item comprises at least one among unique loudness, loudness variation, a frequency, a cycle, sustainability, sound quality, a tone, tone variation, intensity, intensity variation, relative time in inspiratory and expiratory stages, and strength of each diagnostic item.

14. The method according to claim 11, wherein the outputting comprises displaying the diagnosis information on a display.

15. A recording medium for storing a computer program comprising a code which implements a method of controlling an electronic apparatus as a computer readable code, wherein operations of an electronic apparatus that communicates with a plurality of information collection modules for collecting physical condition information at a plurality of points on a body of a user, the operations comprising:
receiving first physical condition information from a first information collection module corresponding to a first point among the plurality of points on the body;
selecting a second point among the plurality of points based on the received first physical condition information; and
receiving second physical condition information from a second information collection module corresponding to the selected second point.
